SEQUENCE LISTING (3 sequences)

SEQ ID NO: 1 - protein, 8 aa, Homo sapiens
NASAPPEE

SEQ ID NO: 2 - protein, 8 aa, Homo sapiens
  VARIANT <1
  VARIANT >8
KPSSPPEE

SEQ ID NO: 3 - protein, 8 aa, Homo sapiens
  VARIANT <1
  VARIANT >8
KPSSPPEE

(19) United States
(12) Patent Application Publication
(10) Pub. No.: US 2023/0272103 A1
(43) Pub. Date: Aug. 31, 2023

(54) CD44-MODULATING COMPOSITIONS FOR METHODS FOR TREATING CANCERS AND ASCITES

(71) Applicant: Splash Pharmaceuticals, Inc., San Diego, CA (US)

(72) Inventors: Malcolm Finlayson, San Diego, CA (US); David Nelson, San Diego, CA (US)

(21) Appl. No.: 18/058,673

(22) Filed: Nov. 23, 2022

Related U.S. Application Data
(63) Continuation-in-part of application No. 16/894,439, filed on Jun. 5, 2020, now abandoned.
Application No. 16/894,439 is a continuation-in-part of application No. 16/072,134, filed on Jul. 23, 2018, now abandoned.
(60) Provisional application No. 63/306,887, filed on Feb. 4, 2022.
(60) Provisional application No. 62/858,781, filed on Jun. 7, 2019.
(60) Provisional application No. 62/290,306, filed on Feb. 2, 2016.
(60) Provisional application No. 62/314,867, filed on Mar. 29, 2016.
(60) Provisional application No. 62/368,964, filed on Jul. 29, 2016.

(51) Int. Cl. C07K 16/28; A61P 35/00; A61K 39/395; A61K 31/337

(52) U.S. Cl. CPC C07K 16/2884; A61P 35/00; A61K 39/3955; A61K 31/337; A61K 2039/505

(57) ABSTRACT

Methods and CD44-modulating peptide compositions for treating or ameliorating ascites, for treating or ameliorating a tumor or cancer, wherein the compositions feature binding moieties such as monoclonal antibodies or fragments thereof specific for CD44 that inhibit CD44 activity, or CD44-modulating peptides that bind to and modulate activity of CD44, optionally featuring a pharmaceutically acceptable carrier. The binding moieties herein may be used with a secondary therapy. In some embodiments, the monoclonal antibody or fragment thereof binds to at least a portion of the cryptic region of CD44.

### CROSS-REFERENCES TO RELATED APPLICATIONS

This application is a non-provisional and claims benefit of U.S. Provisional Patent Application No. 63/306,887 filed Feb. 4, 2022, the specification(s) of which is/are incorporated herein in their entirety by reference.

This application is also a continuation-in-part and claims benefit of U.S. patent application Ser. No. 16/894,439 filed Jun. 5, 2020, the specification(s) of which is/are incorporated herein in their entirety by reference.

U.S. patent application Ser. No. 16/894,439 is a non-provisional and claims benefit of U.S. Provisional Patent Application No. 62/858,781, filed Jun. 7, 2019, the specification of which is incorporated herein in its entirety by reference.

U.S. patent application Ser. No. 16/894,439 is also a continuation-in-part and claims benefit of U.S. patent application Ser. No. 16/072,134, filed Jul. 23, 2018, which is a 371 application and claims benefit of PCT/US2017/015754, filed Jan. 31, 2017, which claims benefit of U.S. Provisional Patent Application No. 62/290,306, filed Feb. 2, 2016, U.S. Provisional Patent Application No. 62/314,867, filed Mar. 29, 2016, and U.S. Provisional Patent Application No. 62/368,964, filed Jul. 29, 2016, the specification(s) of which is/are incorporated herein in their entirety by reference.

### REFERENCE TO AN ELECTRONIC SEQUENCE LISTING

The contents of the electronic sequence listing (SPLASH_19_01_PCT_US_CIP2_Sequence_Listing.xml; Size: 4.184 bytes; and Date of Creation: Nov. 23, 2022) is herein incorporated by reference in its entirety.

### BACKGROUND OF THE INVENTION

### Field of the Invention

The present invention relates to binding moieties such as antibodies or antibody fragments, including monoclonal antibodies or monoclonal antibody fragments, for modulating CD44 activity. The present invention also relates to methods and compositions for treating diseases, conditions, aberrant cellular processes, and symptoms thereof based using compositions that modulate CD44 activity, e.g., bind to a region of CD44.

### Background Art

Strategies are needed that more effectively treat tumors and inhibit the metastatic process in cancer patients. The present invention provides compositions, e.g., polypeptides such as antibodies or fragments thereof, including monoclonal antibodies or fragments thereof, that bind to and inhibit CD44 activity, e.g., modulating drug efflux via CD44. For example, the monoclonal antibodies (or fragments thereof) may be specific for the cryptic region of CD44, or a portion thereof. The present invention is not limited to monoclonal antibodies or fragments thereof and includes CD44-modulating drugs, peptides, or other compositions.

### BRIEF SUMMARY OF THE INVENTION

Provided herein are compositions comprising CD44-modulating polypeptides, e.g., binding moieties such as but not limited to monoclonal antibodies that modulate CD44 activity, e.g., anti-CD44 monoclonal antibodies. The anti-CD44 binding moieties may inhibit activity of CD44, e.g., inhibit efflux of drugs or other molecules or agents in the cell. In some embodiments, the anti-CD44 binding moieties, e.g., the anti-CD44 monoclonal antibodies or fragments thereof, bind to at least a portion of the cryptic region of CD44.

Also provided herein are compositions comprising CD44-modulating binding moieties described herein (e.g., anti-CD44 monoclonal antibodies or fragments thereof, CD44-modulating peptides, etc.) and a secondary therapy, such as but not limited to an anti-cancer agent, radiation therapy, etc.

The compositions comprising CD44-modulating polypeptides (e.g., anti-CD44 monoclonal antibodies, CD44 modulating drugs or peptides) may be useful in methods of treating tumors or cancers as set forth herein.

The compositions comprising CD44-modulating polypeptides (e.g., anti-CD44 monoclonal antibodies, CD44-modulating peptides, drugs, etc.) may be useful in methods of treating or ameliorating ascites as set forth herein.

For example, the present invention features a composition comprising an anti-CD44 monoclonal antibody or a fragment thereof. The composition may be effective for treating a cancer or tumor, such as but not limited to ovarian cancer, breast cancer, lung cancer, prostate cancer, and colon cancer. In some embodiments, the antibody or fragment binds to at least a portion of a cryptic region of CD44 according to SEQ ID NO: 1 (NASAPPEE). In some embodiments, the antibody or fragment thereof binds to and inhibits activity of CD44. In some embodiments, the composition further comprises a pharmaceutically acceptable excipient for administration.

The present invention also features a method of treating a tumor in a patient, said method comprising administering a therapeutic amount of an anti-CD44 antibody or fragment thereof that binds to and inhibits activity of CD44. In some embodiments, the anti-CD44 antibody or fragment thereof binds to at least a portion of a cryptic region of CD44 according to SEQ ID NO: 1 (NASAPPEE). In some embodiments, the method further comprises co-administering a therapeutic amount of a secondary therapy. In some embodiments, the secondary therapy comprises an antimicrotubule agent. In some embodiments, the antimicrotubule agent comprises paclitaxel, a paclitaxel analog, docetacel, vinorelbine, vinblastine, cabaziltaxel, or a combination thereof.

The present invention also features a method of treating, ameliorating, or preventing ascites in a patient in need thereof, said method comprising administering a therapeutically effective amount of a CD44-modulating polypeptide of the present invention. In some embodiments, the CD44-modulating polypeptide comprises SEQ ID NO: 2 or SEQ ID NO: 3. In some embodiments, the CD44-modulating polypeptide comprises an anti-CD44 monoclonal antibody or a fragment thereof that binds to and inhibits activity of CD44. In some embodiments, the anti-CD44 antibody or fragment thereof binds to at least a portion of a cryptic region of CD44 according to SEQ ID NO: 1 (NASAPPEE). In some embodiments, SEQ ID NO: 2 is administered in a dose of 150 mg twice daily. In some embodiments, SEQ ID NO: 2 is administered in a dose of 150 mg once daily. In some embodiments, SEQ ID NO: 2 or SEQ ID NO: 3 is co-administered with a chemotherapeutic agent for also treating a cancer or tumor. In some embodiments, the chemotherapeutic agent comprises paclitaxel, a paclitaxel analog, docetacel, vinorelbine, vinblastine, cabaziltaxel, or a combination thereof.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows CD44 and an 8 amino acid peptide that binds to CD44 and modulates its actions.

FIG. 2 shows tumor response and TP53 alterations or expression, including progression free survival (PFS).

FIG. 3 shows progression free survival estimates.

FIG. 4 shows the subjective results related to ascites.

### DETAILED DESCRIPTION OF THE INVENTION

The terms “polypeptide” and “protein” are used interchangeably herein and refer to any molecule that includes at least 2 or more amino acids.

As used herein, “administering” and the like refer to the act physically delivering a composition or other therapy (e.g. a radiation therapy) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. Radiation therapy can be administered using techniques described herein, including for example, external beam radiation or brachytherapy. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

The term “coadministration” refers to administration of two or more agents (e.g., a polypeptide described herein and another active agent such as an anti-cancer agent or other therapy (e.g. a radiation therapy) described herein). The timing of coadministration depends in part on the combination and compositions or other therapies administered and can include administration at the same time, just prior to, or just after the administration of one or more additional therapies, for example cancer therapies such as chemotherapy, hormonal therapy, radiotherapy, or immunotherapy. Coadministration is meant to include simultaneous or sequential administration of a composition or therapy individually or in combination (more than one polypeptide described herein or an anti-cancer agent described herein or radiation therapy as described herein). Coadministration can include administration of two or more agents where the agents are optionally combined with other active substances (e.g., to reduce metabolic degradation). The polypeptides, anti-cancer agents and radiation therapies described herein can be used in combination with one another, with other active agents known to be useful in treating a disease associated with cells expressing a particular kinase as described herein, or with adjunctive agents that cannot be effective alone, but can contribute to the efficacy of the active agent.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances the term patient refers to a human.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all precancerous and cancerous cells and tissues resulting in abnormal tissue growth. “Neoplastic,” as used herein, refers to any form of dysregulated or unregulated cell growth, whether malignant or benign, resulting in abnormal tissue growth.

An improvement in the cancer or cancer-related disease can be characterized as a complete or partial response. Complete response refers to an absence of clinically detectable disease with normalization of any previously abnormal radiographic studies, bone marrow, and cerebrospinal fluid (CSF) or abnormal monoclonal protein measurements. Partial response refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

An improvement in the cancer or cancer-related disease can be characterized as no evidence of disease (NED).

A refractory, resistant, or persistent cancer refers to a circumstance where patients, even after intensive treatment, have residual cancer cells (e.g., leukemia cells, lymphoma cells, circulating tumor cells or cancer stem cells) in their lymphatic system, blood and/or blood forming tissues (e.g., marrow).

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The term “preventing” refers to the treatment with or administration of a polypeptide or agent (e.g. anti-cancer agent described herein) provided herein, with or without other additional active agent (e.g. an anti-cancer agent), prior to the onset of symptoms, particularly to patients at risk of cancer and/or other disorders described herein. The term also refers to coadministration of a polypeptide with other therapies including radiation therapies as described herein. It should be understood that the polypeptides described herein can be coadministered with one or more anti-cancer agents and radiation therapies described herein. The term prevention includes the inhibition or reduction of a symptom of the particular disease, as well as a reduced incidence of a symptom of the particular disease (e.g. by comparison to historical data for a given subject, or population data for similar subjects). Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

A prophylactically effective amount of a polypeptide or agent (e.g. an anti-cancer agent described herein) means an amount of therapeutic agent, alone or in combination with other agents, which provides a prophylactic benefit in the inhibition or reduced incidence of a symptom of a disease or recurrence of a disease. The term also refers to coadministration of a polypeptide described herein with other therapies including radiation therapies as described herein. The term prophylactically effective amount can encompass an amount that improves overall prophylaxis or enhances the prophylactic efficacy of another prophylactic agent.

The term “effective amount” as used herein refers to the amount of a therapy (e.g., a composition or radiation therapy provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” is an amount sufficient to provide a therapeutic benefit in the treatment or management of a cancer, or to delay or minimize one or more symptoms associated with the presence of the cancer. The term “therapeutically effective amount” can encompass an amount that improves overall therapy, reduces or avoids symptoms or causes of cancer, or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, and/or other therapies useful in the prevention, management, treatment and/or amelioration of a given disease, disorder or condition known to one of skill in the art such as medical personnel.

A regimen is a protocol for dosing and timing the administration of one or more therapies (e.g., combinations described herein, another active agent such as for example an anti-cancer agent described herein, or a radiation therapy described herein) for treating a disease, disorder, or condition described herein. A regimen can include periods of active administration and periods of rest as known in the art. Active administration periods include administration of combinations and compositions described herein and the duration of time of efficacy of such combinations, compositions, and radiation therapies. Rest periods of regimens described herein include a period of time in which no agent (e.g., a polypeptide described herein or an anti-cancer agent described herein) is actively administered, and in certain instances, includes time periods where the efficacy of such agents can be minimal. Rest periods of regimens described herein can include a period of time in which no radiation therapy is actively administered. Combination of active administration and rest in regimens described herein can increase the efficacy and/or duration of administration of the combinations and compositions described herein.

The term “pharmaceutically acceptable” as used herein refers to physiologically acceptable compounds, agents, or ingredients recognized by a regulatory agency of the Federal or state government, or another governmental agency with authorization for such approval, or and an agent listed in the U.S. Pharmacopeia, European Pharmacopeia or other generally recognized Pharmacopeia for use in animals, and more particularly in humans.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, sucrose, glucose, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6^{th} Ed., The Pharmaceutical Press, RPS Publishing (2009). The terms binder, filler, disintegrant, and lubricant are used in accordance with the plain and ordinary meaning within the art.

In certain embodiments, a pharmaceutically acceptable excipient may be incompatible (e.g., cross-reacts) with other excipients or active agents described herein. In some embodiments, magnesium stearate, croscarmellose sodium, lactose, excipients comprising Mg, Ca, K, Li, or Nucleic acid, acesulfame potassium, ammonium alginate, calcium acetate, calcium alginate, calcium carbonate, calcium chloride, calcium lactate, calcium phosphate, calcium silicate, calcium stearate, calcium sulfate, carboxymethylcellulose calcium, carboxymethylcellulose sodium, docusate sodium, glycine, kaolin, magnesium aluminum silicate, magnesium carbonate, magnesium oxide, magnesium silicate, magnesium trisilicate, polacrilin potassium, polymethacrylates, potassium alginate, potassium benzoate, potassium bicarbonate, potassium chloride, potassium citrate, sodium alginate, sodium benzoate, sodium chloride, sodium lauryl sulfate, sodium starch glycolate, sodium stearyl fumarate, sulfobutylether beta-cyclodextrin, sodium stearate, talc, or zinc stearate are incompatible in the dosage forms described herein.

The term “anti-cancer agent” is used in accordance with its plain ordinary meaning and refers to a composition having anti-neoplastic properties or the ability to inhibit the growth or proliferation of cells. In certain embodiments, an anti-cancer agent is a chemotherapeutic. In certain embodiments, an anti-cancer agent is an agent identified herein having utility in methods of treating cancer. In certain embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” or “cancer therapy” refers to a therapy or regimen that includes administration of a combination, chemotherapeutic, or anti-cancer agent described herein.

The term “radiation therapy” is used in accordance with its plain ordinary meaning and refers to the medical use of radiation in the treatment of cancer. Preferably, the medical use of radiation in the treatment of cancer results in the killing of cancer cells in the subject. A variety of radiation therapies can be used in accordance with the present disclosure, examples of which are provided herein.

The term “peptidomimetic,” as used herein, means a peptide-like molecule that has the activity of the peptide upon which it is structurally based. Such peptidomimetics include chemically modified peptides, peptide-like molecules containing non-naturally occurring amino acids, and peptoids, and have an activity such as the selective homing activity of the peptide upon which the peptidomimetic is derived (see, for example, Goodman and Ro, Peptidomimetics for Drug Design, in “Burger's Medicinal Chemistry and Drug Discovery” Vol. 1 (ed. M.E. Wolff; John Wiley & Sons (1995), pages 803-861).

The term “prodrug” refers to a compound or polypeptide that is made more active in vivo through metabolism of a precursor drug. CD44-modulating polypeptides described herein can exist as prodrugs, as described in, for example, Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the polypeptides described herein are structurally modified forms of the peptide that readily undergo chemical changes under physiological conditions to provide the active polypeptide. Additionally, prodrugs can be converted to the active polypeptide by chemical or biochemical methods in an ex vivo environment.

A PD-1 inhibitor refers to a moiety (e.g., compound, nucleic acid, polypeptide, antibody) that decreases, inhibits, blocks, abrogates or interferes with the activity or expression of PD-1 (e.g., Programmed Cell Death Protein 1; PD-1 (CD279); GI: 145559515), including variants, isoforms, species homologs of human PD-1 (e.g., mouse) and analogs that have at least one common epitope with PD-1. A PD-1 inhibitor includes molecules and macromolecules such as, for example, compounds, nucleic acids, polypeptides, antibodies, peptibodies, diabodies, minibodies, nanobodies, single-chain variable fragments (ScFv), and functional fragments or variants thereof. Thus, a PD-1 inhibitor as used herein refers to any moiety that antagonizes PD-1 activity or expression. PD-1 inhibitor efficacy can be measured, for example, by its inhibitor concentration at 50% (half-maximal inhibitor concentration or IC50). PD-1 inhibitors include exemplary compounds and compositions described herein. A PD-1 antibody refers to a PD-1 inhibitor which is a monoclonal or polyclonal antibody as described herein.

### DETAILED DESCRIPTION OF THE INVENTION

All patents, applications, published applications and other publications are incorporated by reference in their entirety and for all purposes. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts. Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

The present invention features CD44-modulating polypeptides, e.g., monoclonal antibodies directed to CD44, e.g., anti-CD44 antibodies. A “CD44-modulating polypeptide” refers to a polypeptide that binds to CD44 and modulates its activity (e.g., signaling activity). A CD44-modulating polypeptide can be a polypeptide sequence described herein, e.g., Ac-KPSSPPEE-NH₂ (SEQ ID NO: 2), a polyclonal or monoclonal antibody that specifically binds to CD44 and inhibits its downstream signaling activity, etc. In some embodiments, the antibody disrupts or inhibits signaling activity of a CD44 dependent co-receptor. In certain instances the CD44 dependent co-receptor is a receptor tyrosine kinase (RTK) such as, for example, Met, Ran, or VEGFR. In some embodiments, the antibody disrupts CD44 co-receptor function or association of a CD44 co-receptor with CD44 or another signaling protein. In one embodiment, a CD44-modulating polypeptide described herein binds to CD44 and inhibits CD44 signaling activity or association with one or more ABC transporters. The ABC transporter can be a multidrug resistant protein (e.g., MDR1).

Methods for producing antibodies, such as monoclonal antibodies, are well known to one of ordinary skill in the art. Methods of producing polyclonal and monoclonal antibodies are known to those of skill in the art and described in the scientific and patent literature, see, e.g., Harlow and Lane. Antibodies. A Laboratory Manual, Cold Spring Harbor Laboratory Press (1989); Hammerling, et al., in: Monoclonal Antibodies and T-Cell Hybridomas 563-681, Elsevier, N.Y. (1981); Harlow et al. Using Antibodies. A Laboratory Manual, Cold Spring Harbor Laboratory Press (1999); and Antibody Engineering: A Practical Guide, Borrebaeck Ed., W.H. Freeman and Co., Publishers, New York, pp. 103-120 (1991); Coligan, Current Protocols in Immunology, Wiley/Green, New York (1991); Stites eds. Basic and Clinical Immunology (7th ed.) Lange Medical Publications, Los Altos, Calif., and references cited therein (Stites); Goding, Monoclonal Antibodies. Principles and Practice (2nd ed.) Academic Press, New York, N.Y. (1986); and Kohler, Nature 256:495 (1975)). Suchtechniques include selection of antibodies from libraries of recombinant antibodies displayed in phage or similar on cells. See, Huse, Science 246:1275 (1989) and Ward, Nature 341:544 (1989). Recombinant antibodies can be expressed by transient or stable expression vectors in mammalian cells, as in Norder haug, J. Immunol. Methods 204:77-87 (1997). In this invention, an antibody also embraces an active fragment thereof. An active fragment means a fragment of an antibody having activity of antigen-antibody reaction. Specifically named, these are active fragments, such as F(ab′), Fab′, Fab, and Fv. For example, F(ab′) results if the antibody of this invention is digested with pepsin, and Fab results if digested with papain. Fab′ results if F(ab′) is reduced with a reagent such as 2-mercaptoethanol and alkylated with monoiodoacetic acid. Fv is a mono active fragment where the variable region of heavy chain and the variable region of light chain are connected with a linker. A chimeric antibody is obtained by conserving these active fragments and substituting the fragments of another animal for the fragments other than these active fragments. In particular, humanized antibodies are included.

In certain embodiments, the CD44-modulating polypeptides have anti-migratory activity on cancer cells. In certain embodiments, the CD44-modulating polypeptides have anti-invasive activity on cancer cells. In certain embodiments, the CD44-modulating polypeptides have anti-metastatic activity on cancer cells.

As disclosed herein, the present invention provides a composition comprising an anti-CD44 monoclonal antibody or a fragment thereof. The composition may be effective for treating a tumor, a cancer, or other tumor-like disease, or tumor-associated condition. In some embodiments, the composition is effective for treating ovarian cancer. In some embodiments, the composition is effective for treating breast cancer. In some embodiments, the composition is effective for treating lung cancer. The present invention is not limited to the particular cancers or tumors disclosed herein. For example, in some embodiments, the composition is effective for treating or ameliorating ascites.

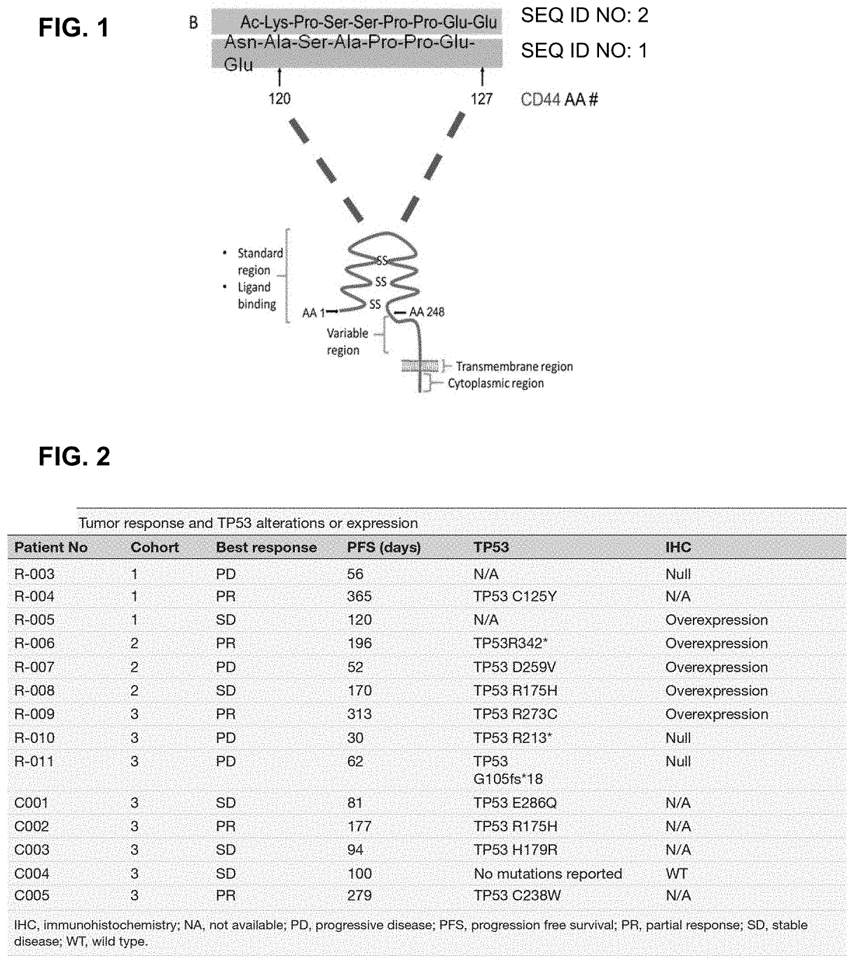

In some embodiments, the anti-CD44 binding moiety, e.g., the anti-CD44 monoclonal antibody or fragment thereof, binds to at least a portion of the cryptic region of CD44. As used herein, the cryptic region of CD44 is aa 120-127 or NASAPPEE (SEQ ID NO: 1), as shown in FIG. 1. In some embodiments, the anti-CD44 monoclonal antibody or fragment thereof binds to at least the first amino acid. In some embodiments, the anti-CD44 monoclonal antibody or fragment thereof binds to at least the first two amino acids. In some embodiments, the anti-CD44 monoclonal antibody or fragment thereof binds to at least the first three amino acids. In some embodiments, the anti-CD44 monoclonal antibody or fragment thereof binds to at least the first four amino acids. In some embodiments, the anti-CD44 monoclonal antibody or fragment thereof binds to at least the first five amino acids. In some embodiments, the anti-CD44 monoclonal antibody or fragment thereof binds to at least the first six amino acids. In some embodiments, the anti-CD44 monoclonal antibody or fragment thereof binds to at least the first seven amino acids. In some embodiments, the anti-CD44 monoclonal antibody or fragment thereof binds to all eight of the amino acids. In some embodiments, the anti-CD44 monoclonal antibody or fragment thereof binds to at least the last seven amino acids. In some embodiments, the anti-CD44 monoclonal antibody or fragment thereof binds to at least the last six amino acids. In some embodiments, the anti-CD44 monoclonal antibody or fragment thereof binds to at least the last five amino acids. In some embodiments, the anti-CD44 monoclonal antibody or fragment thereof binds to at least the last four amino acids. In some embodiments, the anti-CD44 monoclonal antibody or fragment thereof binds to at least the last three amino acids. In some embodiments, the anti-CD44 monoclonal antibody or fragment thereof binds to at least the last two amino acids. In some embodiments, the anti-CD44 monoclonal antibody or fragment thereof binds to at least the last amino acid.

The present invention also provides a CD44-modulating peptide according to SEQ ID NO: 2. As shown in FIG. 1, the peptide according to SEQ ID NO: 2 can bind to the cryptic region of CD44 (SEQ ID NO: 1).

The present invention also provides a pharmaceutical composition comprising an anti-CD44 monoclonal antibody and a pharmaceutically acceptable excipient for administration. The present invention also provides a pharmaceutical composition comprising a peptide according to SEQ ID NO: 2 and a pharmaceutically acceptable excipient for administration.

The present invention also provides a composition comprising an anti-CD44 binding fragment.

The present invention also provides a composition comprising a monoclonal anti-CD44 binding fragment.

The present invention also provides the use of an anti-CD44 antibody or an antigen binding fragment thereof for treating a tumor in a patient.

The present invention also provides the use of an anti-CD44 antibody or an antigen binding fragment thereof, in combination with a secondary therapy, for treating a tumor in a patient.

The present invention also features methods and compositions (e.g., CD44 modulating peptides such as SEQ ID NO: 2, anti-CD44 antibodies or fragments thereof, etc.) for treating or ameliorating ascites.

The present invention also includes compositions comprising anti-CD44 antibodies (e.g., monoclonal antibodies) and one or more secondary therapies. Non-limiting examples of secondary therapies include radiation and anti-cancer agents, e.g, as described below.

The anti-cancer agent used in combination with a polypeptide described herein can be a taxane, a platinum agent, anthracyclin, or a checkpoint inhibitor. The anti-cancer agent can be taxane. The anti-cancer agent can be taxol, taxotere, paclitaxel, or cabazitaxel. The anti-cancer agent can be a platinum agent such as, for example, cisplatin, carboplatin, oxaliplatin, satraplatin, picoplatin, nedaplatin, or triplatin. In certain instance the platinum agent can be carboplatin or oxaliplatin. The anti-cancer agent can be paclitaxel, a paclitaxel analogue, docetaxel, cabaziltaxel, doxorubicin, a checkpoint inhibitor, cisplatin, oxaliplatin, carboplatin, methotrexate, or a PARP inhibitor. In certain embodiments, the anti-cancer agent can be paclitaxel, a paclitaxel analogue, docetaxel, cabaziltaxel, doxorubicin, a checkpoint inhibitor, cisplatin, oxaliplatin, carboplatin, methotrexate, or a PARP inhibitor. In another example the anti-cancer agent can be methotrexate. The anti-cancer agent can be an anthracyclin agent. The anti-cancer agent can be amrubicin, daunorubicin, epirubicin, idarubicin, doxorubicin, pirarubicin, or valrubicin. The cancer treated can be resistant to treatment with an anthracyclin agent. For example, the anti-cancer agent can be amrubicin, daunorubicin, epirubicin, idarubicin, doxorubicin, pirarubicin, or valrubicin. The anti-cancer agent can be a PARP inhibitor. The PARP inhibitor can inhibit one or both of PARP1 and PARP2. The PARP inhibitor can be talazoparib (BMN 673), olaparib, rucaparib, iniparib, or veliparib.

The anti-cancer agents can include any known class of anti-cancer agents such as, for example, operations, alkylating agents, antimetabolites, anthracyclines, campothecins, vinca alkaloids, taxanes or platinums, as well as other antineoplastic agents known in the art. Such anti-cancer agent and antineoplastic agent classifications are known in the art and used in accordance with their plain and ordinary meaning. Such anti-cancer agents and anti-cancer agent classes can in certain instances can provide anti-cancer activity in combination with each other. Exemplary anti-cancer agents include but are not limited to: ABRAXANE; abiraterone; ace-11; aclarubicin; acivicin; acodazole hydrochloride; acronine; actinomycin; acylfulvene; adecypenol; adozelesin; adriamycin; aldesleukin; all trans-retinoic acid (ATRA); altretamine; ambamustine; ambomycin; ametantrone acetate; amidox; amifostine; aminoglutethimide; aminolevulinic acid; amrubicin; amsacrine; anagrelide; anastrozole; andrographolide; antarelix; anthramycin; aphidicolin glycinate; apurinic acid; ara-CDP-DL-PTBA; arginine deaminase; ARRY-162; ARRY-300; ARRY-142266; AS703026; asparaginase; asperlin; asulacrine; atamestane; atrimustine; AVASTIN; axinastatin 1; axinastatin 2; axinastatin 3; azasetron; azatoxin; azatyrosine; azacitidine; AZD8330; azetepa; azotomycin; balanol; batimastat; BAY 11-7082; BAY 43-9006; BAY 869766; bendamustine; benzochlorins; benzodepa; benzoylstaurosporine; beta-alethine; betaclamycin B; betulinic acid; b-FGF inhibitor; bicalutamide; bisantrene; bisaziridinylspermine; bisnafide; bisnafide dimesylate; bistratene A; bisantrene hydrochloride; bleomycin; bleomycin sulfate; busulfan; bizelesin; breflate; bortezomib; brequinar sodium; bropirimine; budotitane; buthionine sulfoximine; bryostatin; cactinomycin; calusterone; calcipotriol; calphostin C; camptothecin derivatives; capecitabine; carboxamide-amino-triazole; carboxyamidotriazole; CaRest M3; CARN 700; caracemide; carbetimer; carboplatin; carmustine; carubicin hydrochloride; carzelesin; castanospermine; cecropin B; cedefmgol; celecoxib; cetrorelix; chlorins; chloroquinoxaline sulfonamide; cicaprost; chlorambucil; Chlorofusin; cirolemycin; cisplatin; CI-1040; cis-porphyrin; cladribine; clomifene analogues; clotrimazole; collismycin A; collismycin B; combretastatin A4; combretastatin analogue; conagenin; crambescidin 816; crisnatol; crisnatol mesylate; cryptophycin 8; cryptophycin A derivatives; curacin A; cyclopentanthraquinones; cycloplatam; cypemycin; cyclophosphamide; cytarabine; cytarabine ocfosfate; cytolytic factor; cytostatin; dacarbazine; dactinomycin; daunorubicin; daunorubicin hydrochloride; decarbazine; dacliximab; dasatinib; decitabine; dehydrodidemnin B; deslorelin; dexamethasone; dexifosfamide; dexrazoxane; dexverapamil; dexormaplatin; dezaguanine; dezaguanine mesylate; diaziquone; didemnin B; didox; diethylnorspermine; dihydro 5 azacytidine; dihydrotaxol; 9-dioxamycin; diphenyl spiromustine; docosanol; dolasetron; docetaxel; doxorubicin; doxorubicin hydrochloride; doxifluridine; droloxifene; droloxifene citrate; dromostanolone propionate; dronabinol; duazomycin; duocarmycin SA; ebselen; ecomustine; edelfosine; edrecolomab; edatrexate; eflornithine hydrochloride; eflornithine; elemene; emitefur; elsamitrucin; enloplatin; enpromate; epipropidine; epirubicin; epirubicin hydrochloride; epristeride; erbulozole; eribulin; esorubicin hydrochloride; estramustine; estramustine phosphate sodium; etanidazole; etoposide; etoposide phosphate; etoprine; exemestane; fadrozole; fadrozole hydrochloride; fazarabine; fenretinide; filgrastim; finasteride; flavopiridol; flezelastine; fluasterone; floxuridine; fludarabine phosphate; fludarabine; fluorodaunorubicin hydrochloride; forfenimex; formestane; fluorouracil; floxouridine; flurocitabine; fosquidone; fostriecin sodium; fostriecin; fotemustine; gadolinium texaphyrin; gallium nitrate; galocitabine; ganirelix; gelatinase inhibitors; gemcitabine; geldanamycin; gossyphol; GDC-0973; GSK1120212/trametinib; herceptin; hydroxyurea; hepsulfam; heregulin; hexamethylene bisacetamide; hypericin; ibandronic acid; ibrutinib; idarubicin; idarubicin hydrochloride; ifosfamide; canfosfamide; ilmofosine; iproplatin; idoxifene; idramantone; ilmofosine; ilomastat; imidazoacridones; imatinib (e.g., GLEEVEC); imiquimod; iniparib (BSI 201); iobenguane; iododoxorubicin; ipomeanol; irinotecan; irinotecan hydrochloride; irsogladine; isobengazole; isohomohalicondrin B; itasetron; iimofosine; interleukin IL-2 (including recombinant interleukin II; or rIL.sub.2); interferon alfa-2a; interferon alfa-2b; interferon alfa-n1; interferon alfa-n3; interferon beta-1a; interferon gamma- 1lb; jasplakinolide; kahalalide F; lamellarin N triacetate; lanreotide; leinamycin; lenograstim; lentinan sulfate; leptolstatin; letrozole; leuprorelin; levamisole; lenalidomide; lenvatinib; liarozole; lissoclinamide 7; lobaplatin; lombricine; lometrexol; lonidamine; losoxantrone; lovastatin; loxoribine; lurtotecan; lutetium texaphyrin; lysofylline; lanreotide acetate; lapatinib; letrozole; leucovorin; leuprolide acetate; liarozole hydrochloride; lometrexol sodium; lomustine; losoxantrone hydrochloride; pomalidomide; LY294002; maitansine; mannostatin A; marimastat; masoprocol; maspin; matrilysin inhibitors; menogaril; merbarone; meterelin; methioninase; metoclopramide; MIF inhibitor; mifepristone; miltefosine; mirimostim; mitoguazone; mitolactol; mitonafide; mitoxantrone; mofarotene; molgramostim; mopidamol; mycaperoxide B; myriaporone; maytansine; mechlorethamine hydrochloride; megestrol acetate; melengestrol acetate; melphalan; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nafarelin; nagrestip; napavin; naphterpin; nartograstim; nedaplatin; nemorubicin; neridronic acid; nilutamide; nisamycin; nitric oxide modulators; nitroxide antioxidant; nitrullyn; nococdazole; nogalamycin; oblimersen (GENASENSE); octreotide; okicenone; olaparib (LYNPARZA); oligonucleotides; onapristone; ondansetron; oracin; oral cytokine inducer; ormaplatin; oxisuran; oxaloplatin; osaterone; oxaliplatin; oxaunomycin; palauamine; palmitoylrhizoxin; pamidronic acid; panaxytriol; panomifene; parabactin; PARP (polyADP ribose polymerase) inhibitors; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; porfiromycin; prednisone; prostaglandin J2; pyrazoloacridine; paclitaxel; PD035901; PD184352; PD318026; PD98059; peliomycin; pentamustine; peplomycin sulfate; PKC412; pipobroman; piposulfan; piroxantrone hydrochloride; plicamycin; plomestane; podophyllotoxin; polyphenol E; porfimer sodium; porfiromycin; prednimustine; procarbazine; procarbazine hydrochloride; puromycin; puromycin hydrochloride; pyrazofurin; raltitrexed; ramosetron; retelliptine demethylated; rhizoxin; rituximab; RII retinamide; rogletimide; rohitukine; romurtide; roquinimex; rubiginone B1; ruboxyl; riboprine; romidepsin; rucaparib; safingol; safmgol hydrochloride; saintopin; sarcophytol A; sargramostim; semustine; sizofiran; sobuzoxane; sodium borocaptate; sodium phenylacetate; solverol; sonermin; sorafenib; sunitinib; sparfosic acid; spicamycin D; spiromustine; splenopentin; spongistatin 1; Spongistatin 2; Spongistatin 3; Spongistatin 4; Spongistatin 5; Spongistatin 6; Spongistatin 7; Spongistatin 8; and Spongistatin 9; squalamine; stipiamide; stromelysin inhibitors; sulfinosine; suradista; suramin; swainsonine; SB239063; selumetinib/AZD6244; simtrazene; SP600125; sparfosate sodium; sparsomycin; spirogermanium hydrochloride; spiroplatin; streptonigrin; streptozocin; sulofenur; tallimustine; tamoxifen methiodide; talazoparib (BMN 673); tauromustine; tazarotene; tecogalan sodium; tegafur; tellurapyrylium; temoporfin; temozolomide; teniposide; tetrachlorodecaoxide; tetrazomine; thaliblastine; thiocoraline; thrombopoietin; thymalfasin; thymopoietin receptor agonist; thymotrinan; tirapazamine; titanocene bichloride; topsentin; toremifene; tretinoin; triacetyluridine; triciribine; trimetrexate; triptorelin; tropisetron; turosteride; tyrphostins; talisomycin; TAK-733; taxotere; tegafur; teloxantrone hydrochloride; teroxirone; testolactone; thiamiprine; thioguanine; thiotepa; tiazofurin; tirapazamine; toremifene citrate; trastuzumab; trestolone acetate; triciribine phosphate; trimetrexate; trimetrexate glucuronate; triptorelin; tubulozole hydrochloride; tumor necrosis factor-related apoptosis-inducing ligand (TRAIL); UBC inhibitors; ubenimex; U0126; uracil mustard; uredepa; vapreotide; variolin B; velaresol; veliparib (ABT-888); veramine; verteporfin; vinorelbine; vinxaltine; vitaxin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; wortmannin; XL518; zanoterone; zeniplatin; zilascorb; zinostatin stimalamer; zinostatin; and zorubicin hydrochloride.

Other exemplary anti-cancer agents include Erbulozole (e.g., R-55104); Dolastatin 10 (e.g., DLS-10 and NSC-376128); Mivobulin isethionate (e.g., CI-980); NSC-639829; Discodermolide (e.g., NVP-XX-A-296); ABT-751 (Abbott; e.g., E-7010); Altorhyrtin A; Altorhyrtin C; Cemadotin hydrochloride (e.g., LU-103793 and NSC-D-669356); CEP 9722; Epothilone A; Epothilone B; Epothilone C; Epothilone D; Epothilone E; Epothilone F; Epothilone B N-oxide; Epothilone A N-oxide; 16-aza-epothilone B; 21-aminoepothilone B; 21-hydroxyepothilone D; 26-fluoroepothilone; Auristatin PE (e.g., NSC-654663); Soblidotin (e.g., TZT-1027); LS-4559-P (Pharmacia; e.g., LS-4577); LS-4578 (Pharmacia; e.g., LS-477-P); LS-4477 (Pharmacia); LS-4559 (Pharmacia); RPR-112378 (Aventis); DZ-3358 (Daiichi); FR-182877 (Fujisawa; e.g., WS-9265B); GS-164 (Takeda); GS-198 (Takeda); KAR-2 (Hungarian Academy of Sciences); BSF-223651 (BASF; e.g., ILX-651 and LU-223651); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (e.g., LY-355703); AC-7739 (Ajinomoto; e.g., AVE-8063A and CS-39.HCl); AC-7700 (Ajinomoto; e.g., AVE-8062; AVE-8062A; CS-39-L-Ser.HCl; and RPR-258062A); Vitilevuamide; Tubulysin A; Canadensol; CA-170 (Curis, Inc.); Centaureidin (e.g., NSC-106969); T-138067 (Tularik; e.g., T-67; TL-138067 and TI-138067); COBRA-1 (Parker Hughes Institute; e.g., DDE-261 and WHI-261); H10 (Kansas State University); H16 (Kansas State University); Oncocidin Al (e.g., BTO-956 and DIME); DDE-313 (Parker Hughes Institute); Fijianolide B; Laulimalide; SPA-2 (Parker Hughes Institute); SPA-1 (Parker Hughes Institute; e.g., SPIKET-P); 3-IAABU (Cytoskeleton/Mt. Sinai School of Medicine; e.g., MF-569); Narcosine (e.g., NSC-5366); Nascapine; D-24851 (Asta Medica); A-105972 (Abbott); Hemiasterlin; 3-BAABU (Cytoskeleton/Mt. Sinai School of Medicine; e.g., MF-191); TMPN (Arizona State University);

Vanadocene acetylacetonate; T-138026 (Tularik); Monsatrol; lnanocine (e.g., NSC-698666); 3-IAABE (Cytoskeleton/Mt. Sinai School of Medicine); A-204197 (Abbott); T-607 (Tuiarik; e.g., T-900607); RPR-115781 (Aventis); Eleutherobins (e.g., Desmethyleleutherobin; Desaetyleleutherobin; lsoeleutherobin A; and Z-Eleutherobin); Caribaeoside; Caribaeolin; Halichondrin B; D-64131 (Asta Medica); D-68144 (Asta Medica); Diazonamide A; A-293620 (Abbott); NPI-2350 (Nereus); Taccalonolide A; TUB-245 (Aventis); A-259754 (Abbott); Diozostatin; (-)-Phenylahistin (e.g., NSCL-96F037); D-62638 (Asta Medica); D-62636 (Asta Medica); Myoseverin B; D-43411 (Zentaris; e.g., D-81862); A-289099 (Abbott); A-318315 (Abbott); HTI-286 (e.g., SPA-110; trifluoroacetate salt) (Wyeth); D-82317 (Zentaris); D-82318 (Zentaris); SC-12983 (NCI; Resverastatin phosphate sodium; BPR-OY-007 (National Health Research Institutes); and SSR-250411 (Sanofi)); goserelin; leuprolide; triptolide; homoharringtonine; topotecan; itraconazole; deoxyadenosine; sertraline; pitavastatin; clofazimine; 5-nonyloxytryptamine; vemurafenib; dabrafenib; gefitinib (IRESSA); erlotinib (TARCEVA); cetuximab (ERBITUX); lapatinib (TYKERB); panitumumab (VECTIBIX); vandetanib (CAPRELSA); afatinib/BIBW2992; CI-1033/canertinib; neratinib/HKI-272; CP-724714; TAK-285; AST-1306; ARRY334543; ARRY-380; AG-1478; dacomitinib/PF299804; OSI-420/desmethyl erlotinib; AZD8931; AEE726; pelitinib/EKB-569; CUDC-101; WZ8040; WZ4002; WZ3146; AG-490; XL647; PD153035; 5-azathioprine; 5-aza-2′-deoxycytidine; 17-N-Allylamino-17-Demethoxygeldanamycin (17-AAG); 20-epi-1,25 dihydroxyvitamin D3; 5 ethynyluracil; and BMS-599626.

The anti-cancer agent can be a checkpoint inhibitor. The term “checkpoint inhibitor” refers to molecules that totally or partially reduce, inhibit, interfere with or modulate one or more checkpoint proteins. Without being limited by a particular theory, checkpoint proteins regulate T-cell activation or function. Exemplary checkpoint include CTLA-4 and its ligands CD80 and CD86; PD-1 and its ligands PD-L1 and PD-L2; TIM-3; OX40 (CD134); GITR; CD137; CD40; recombinant human interleukin-15 (rhIL-15); and IDO. These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors can include antibodies or are derived from antibodies. The checkpoint inhibitor can be a CTLA-4 inhibitor. The CTLA-4 inhibitor can be an anti-CTLA-4 antibody. Examples of anti-CTLA-4 antibodies include, but are not limited to, those described in U.S. Pat. Nos: 5,811,097; 5,811,097; 5,855,887; 6,051,227; 6,207,157; 6,682,736; 6,984,720; and 7,605,238, all of which are incorporated herein in their entireties and for all purposes. Exemplary anti-CTLA-4 antibodies include tremelimumab and ipilimumab. Thus, provided herein are methods of treating a cancer by administering a CD44-modulating polypeptide described herein in combination with tremelimumab. In another aspect provided herein are methods of treating cancer described herein by administering a CD44-modulating polypeptide described herein in combination with ipilimumab. The checkpoint inhibitor can be a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties and for all purposes. The checkpoint inhibitor can be a PD-1 inhibitor. The checkpoint inhibitor can be an anti-PD-1 antibody. Exemplary PD-1/PD-L1/PD-L2 inhibitors and antibodies include nivolumab, pembrolizumab, AMP-224, pidilizumab, REGN2810, PDR 001, MEDI0680, durvalumab, avelumab, atezolizumab, BMS-936559, or rHIgM12B7A. The checkpoint inhibitor can be a lymphocyte activation gene-3 (LAG-3) inhibitor. Exemplary LAG-3 inhibitors include IMP321 and BMS-986016. The checkpoint inhibitor can be a B7 inhibitor. In one embodiment, the B7 inhibitor can be a B7-H3 inhibitor or a B7-H4 inhibitor. The B7-H3 inhibitor can be MGA271. In another aspect, the checkpoint inhibitor can be a TIM-3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94). The checkpoint inhibitor can be an 0X40 (CD134) agonist. In one embodiment, the checkpoint inhibitor can be an anti-OX40 antibody., such as for example, MEDI6469. In still another aspect, the checkpoint inhibitor can be a GITR agonist. In one embodiment, the checkpoint inhibitor can be an anti-GITR antibody. In one embodiment, the anti-GITR antibody can be TRX518. In yet another aspect, the checkpoint inhibitor can be a CD137 agonist. In one embodiment, the checkpoint inhibitor can be an anti-CD137 antibody. Exemplary anti-CD137 antibodies include urelumab and PF-05082566. The checkpoint inhibitor can be a CD40 agonist. In one embodiment, the checkpoint inhibitor can be an anti-CD40 antibody such as, for example, CF-870,893. In yet another aspect, the checkpoint inhibitor can be recombinant human interleukin-15 (rhIL-15). In another aspect, the checkpoint inhibitor can be an IDO inhibitor. Exemplary IDO inhibitors include INCB024360 and indoximod.

The present invention also features methods for treating cancer by administering an effective amount of a CD44-modulating polypeptide. In some embodiments, the method features administering an effective amount of a CD44-modulating peptide and a secondary therapeutic, e.g., an anti-cancer agent. In some embodiments, the CD44-modulating polypeptide is according SEQ ID NO: 2 (Ac-NASAPPEE-NH₂). In some embodiments, the CD44-modulating peptide comprises a peptide according to SEQ ID NO: 3 (Ac-KPSSPPEE-NH₂). In some embodiments, the CD44-modulating peptide is the peptide according to SEQ ID NO: 2 or SEQ ID NO: 3 with at least one modification, e.g., post-translational modification. In some embodiments, the CD44-modulating peptide comprises at least 75% of the amino acids of SEQ ID NO: 2 or SEQ ID NO: 3. In some embodiments, the CD44-modulating peptide comprises more than 75% of the amino acids of SEQ ID NO: 2. In some embodiments, one or more amino acids of SEQ ID NO: 2 or SEQ ID NO: 3 is replaced with an analog. In some embodiments, the CD44-modulating peptide comprises at least one glycosylated amino acid. CD44-modulating peptides can include substitution variants, addition variants, or chemical derivatives thereof including peptidomimetics. Other exemplary CD44-modulating peptides include those set forth in U.S. Pat. Nos. 5,994,309; 6,696,416; and 6,963,587 and U.S. Patent Application Publication No. US2009192085, the disclosures of which are incorporated herein in their entirety by reference.

### Example 1

The following is a non-limiting example of the present invention. It is to be understood that said example is not intended to limit the present invention in any way. Equivalents or substitutes are within the scope of the present invention.

As previously discussed, the present invention also features CD44-modulating peptides, e.g., anti-CD44 antibodies or fragments thereof, or a peptide according to SEQ ID NO: 2) for treating or ameliorating ascites.

The following Example describes a phase I, open label, dose escalation study of the safety and tolerability of the combination of SPL-108 (SEQ ID NO: 2) with weekly paclitaxel in patients with platinum resistant CD44+ ovarian, primary peritoneal, or fallopian tube cancer. As will be shown below, a combination of SPL-108 (SEQ ID NO: 2) with weekly paclitaxel was safe and well tolerated. Encouraging antitumor activity was observed, with 72% of patients deriving a clinical benefit. It was also shown that the treatments improved ascites volumes.

Patients with platinum resistant histologically proven epithelial ovarian, primary peritoneal, or fallopian tube cancers and measurable disease according to RECIST (Response Evaluation Criteria in Solid Tumours) version 1.1 were selected. Tumors were tested for CD44 expression for eligibility, defined as strong (+++) or moderate (++) staining in ≥20% of the tumor tissue or diffuse +staining. This was a phase I, open label clinical study of SPL-108 (SEQ ID NO: 2) and paclitaxel using a standard ‘3+3’ dose escalation design, including two different dose levels of eligible patients, followed by expansion cohort. Patients on dose level 1 received SPL-108 (SEQ ID NO: 2) 150 mg subcutaneously every 24 hours with 80 mg/m2 paclitaxel weekly intravenously on days 1, 8, and 15 of a 28 day cycle. Patients on dose level 2 and the subsequent expansion cohort received SPL-108 (SEQ ID NO: 2) 150 mg subcutaneously every 12 hours with 80 mg/m2 paclitaxel weekly intravenously with the same schedule. Progression free survival estimates with 95% confidence intervals (CI) were calculated using complementary log-log transformation of progression free survival estimates. Categorical variables are presented in frequency tables. Laboratory correlates were performed in triplicate and the t test used for significance estimates.

All patients who received at least one cycle of protocol therapy were evaluated for toxicity. Adverse events were assessed weekly according to the National Cancer Institute terminology criteria for Adverse Events (CTCAE) version 4.0. A dose limiting toxicity was defined as an adverse event that met one of the following criteria: (1) treatment related ≥grade 3 non-hematologic toxicity, excluding alopecia, hypersensitivity reactions, and injection site reactions; (2) grade 4 neutropenia for at least 7 days, febrile neutropenia, and thrombocytopenia accompanied by bleeding; or (3) ≥grade 3 hematologic toxicity, excluding anemia and lymphocytopenia. Maximum tolerated dose was defined as the dose below the dose at which at least two of six patients experienced dose limiting toxicities. Patients were evaluated for response of measurable disease using computerized tomography (CT) imaging of the chest/abdomen/pelvis at baseline and after every two cycles of protocol therapy. Progression free survival was defined as the date of registration until disease progression or death, whichever came first (censored by the date of last contact prior to data analysis).

Fourteen of 16 patients had CD44+ tumors and were enrolled in the study. Median age was 60.5 years (45-77). Most patients had high grade serous carcinoma histology (n=12, 86%) and platinum resistant disease (n=12, 86%). Ten patients had received ≤3 prior chemotherapy regimens; four patients had received 4-5 prior regimens. Seven patients (50%) had received prior PARP inhibitor therapy and six (43%) had received prior bevacizumab

Eighty-two treatment cycles were administered with a median of four cycles (range 2-14). Eight patients (57%) received ≥4 cycles. There were no grade 4-5 toxicities. One patient experienced grade 3 peripheral sensory neuropathy attributed to paclitaxel. An additional patient developed a presumed colonic perforation attributed to the study drug, described below (Table 2). There were no injection site reactions or dermatological adverse events and no treatment discontinuations or treatment related deaths. No dose reductions were required. All patients tolerated the maximum planned dose well; no maximum tolerated dose was reached. Six patients completed dose escalation. Eight additional patients completed the expansion phase.

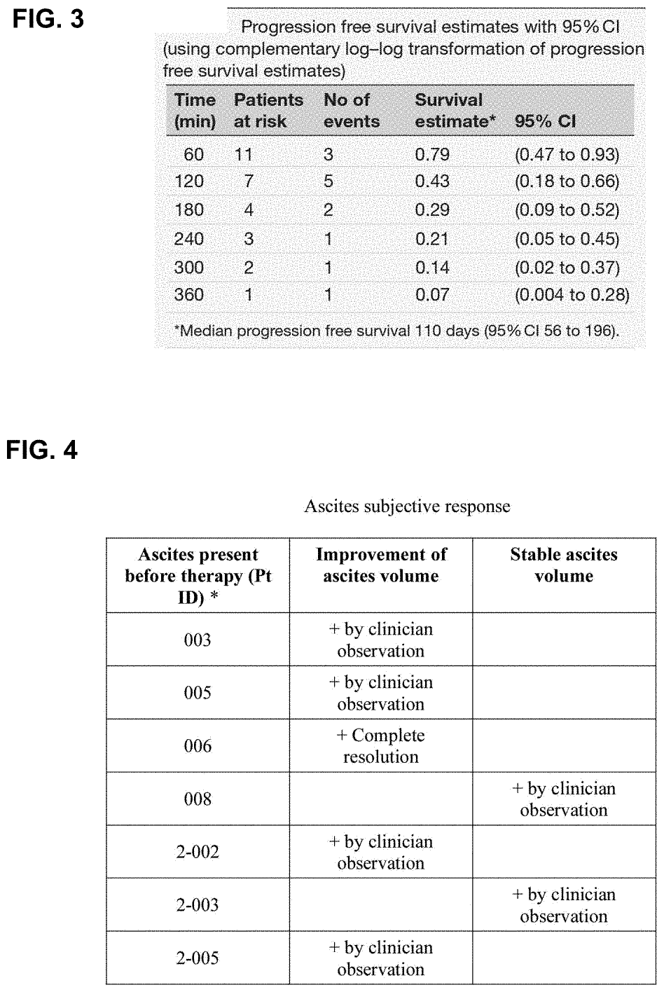

Promising clinical benefit was observed. Median progression free survival was 110 days (95% CI 56 to 196) (see FIG. 2, FIG. 3). The overall response rate was 36%; five patients had a partial response with a median duration of 10 months. Five patients (36%) had stable disease and four (29%) had progression of disease (FIG. 2). All patients with partial response as best response had not been treated with a PARP inhibitor. In contrast, three of four patients who had progression of disease as best response received prior PARP inhibitors as their last therapy prior to the trial. There was a correlation between CA-125 and clinical impression of reduced ascites, however; none met CA-125 criteria of response. Seven patients had ascites at the beginning of therapy; none had progression of ascites and one had complete ascites resolution (FIG. 4). This correlates with our preclinical data, which showed that SPL-108 (SEQ ID NO: 2) had antiangiogenic effects and decreased vascular endothelial growth factor expression in an ovarian cancer cell line.

Eleven patients (79%) had comprehensive genomic profiling using hybrid capture technology. All tumors had low or intermediate mutational burden. No tumors had increased mutation burden or microsatellite instability. One tumor had an MLH1 truncation, but the tumor was microsatellite stable. One patient had a germline BRCA1 mutation, while three patients had somatic BRCA1 mutation.

All four patients with progression of disease had TP53 alterations with loss of function: three nonsense TP53 truncation alterations and one known loss of function. The three tumors that had TP53 truncations showed complete absence of p53 by standard immunohistochemistry, while the tumor with loss of function, but no truncation, had mutant p53 overexpression. Tumors with either a partial response or stable disease had TP53wt (one) and missense gain of function alterations. One tumor that responded with a partial response had a truncation at the end of the molecule, and p53 was likely still functional.

The effect of SPL-108 (SEQ ID NO: 2) on ABCB1 expression was tested using western blot. SPL-108 (SEQ ID NO: 2) reduced total levels of ABCB1 in both BRCA wild type (OVCAR8 and SKOV3) and BRCA2 mutated (PEO1) cells, indicating that SPL-108 (SEQ ID NO: 2) in vitro properties are not cell type specific or dependent on BRCA expression. Furthermore, SPL-108 (SEQ ID NO: 2) is affecting ABCB1 expression, a known downstream target of CD44.

OVCAR8 cells were tested to determine the effect of SPL-108 (SEQ ID NO: 2) on vascular endothelial growth factor as a possible mechanism for the finding. After treatment with SPL-108 (SEQ ID NO: 2), both ABCB1 and vascular endothelial growth factor expression were decreased by 50%. Given that patients who had PARP inhibitors as the last therapy prior to the trial did not respond to the trial therapy, a PARP inhibitor resistant cell line derived from the parent cell line OVCAR8 that had both high P-glycoprotein (ABCB1) and CD44 expression was tested. Western blots showed that treatment with SPL-108 (SEQ ID NO: 2) decreased ABCB1 expression in parental PARP inhibitor sensitive ovarian cancer cell lines by 50% compared with PARP inhibitor resistant cells. These results can inform the inclusion criteria of future clinical trials.

This phase I trial demonstrated that a combination of SPL-108 (SEQ ID NO: 2) and paclitaxel is safe and well tolerated in patients with platinum resistant ovarian cancer. While the maximum tolerated dose was not reached in the present trial, doses such as 150 mg SPL-108 (SEQ ID NO: 2) subcutaneously twice daily may be considered. The present invention is not limited to the doses disclosed herein.

It is understood that modifications which do not substantially affect the activity of the various embodiments of this invention are also included within the definition of the invention provided herein.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed:

1. A composition comprising an anti-CD44 monoclonal antibody or a fragment thereof.

2. The composition of claim 1, wherein the composition is effective for treating ovarian cancer.

3. The composition of claim 1, wherein the composition is effective for treating breast cancer.

4. The composition of claim 1, wherein the composition is effective for treating lung cancer.

5. The composition of claim 1, wherein the antibody or fragment binds to at least a portion of a cryptic region of CD44 according to SEQ ID NO: 1 (NASAPPEE).

6. The composition of claim 5, wherein the antibody or fragment thereof binds to and inhibits activity of CD44.

7. The composition of claim 1, wherein the composition further comprises a pharmaceutically acceptable excipient for administration.

8. A method of treating a tumor in a patient, said method comprising administering a therapeutic amount of an anti-CD44 antibody or fragment thereof that binds to and inhibits activity of CD44.

9. The method of claim 8, wherein the anti-CD44 antibody or fragment thereof binds to at least a portion of a cryptic region of CD44 according to SEQ ID NO: 1 (NASAPPEE).

10. The method of claim 8 further comprising co-administering a therapeutic amount of a secondary therapy.

11. The method of claim 10, wherein the secondary therapy comprises an antimicrotubule agent.

12. The method of claim 11, wherein the antimicrotubule agent comprises paclitaxel, a paclitaxel analog, docetacel, vinorelbine, vinblastine, cabaziltaxel, or a combination thereof.

13. A method of treating, ameliorating, or preventing ascites in a patient in need thereof, said method comprising administering a therapeutically effective amount of a CD44-modulating polypeptide of the present invention.

14. The method of claim 13, wherein the CD44-modulating polypeptide comprises SEQ ID NO: 2 or SEQ ID NO: 3.

15. The method of claim 13, wherein the CD44-modulating polypeptide comprises an anti-CD44 monoclonal antibody or a fragment thereof that binds to and inhibits activity of CD44.

16. The method of claim 13, wherein the anti-CD44 antibody or fragment thereof binds to at least a portion of a cryptic region of CD44 according to SEQ ID NO: 1 (NASAPPEE).

17. The method of claim 14, wherein SEQ ID NO: 2 is administered in a dose of 150 mg twice daily.

18. The method of claim 14, wherein SEQ ID NO: 2 is administered in a dose of 150 mg once daily.

19. The method of claim 14, wherein SEQ ID NO: 2 or SEQ ID NO: 3 is co-administered with a chemotherapeutic agent for also treating a cancer or tumor.

20. The method of claim 19, wherein the chemotherapeutic agent comprises paclitaxel, a paclitaxel analog, docetacel, vinorelbine, vinblastine, cabaziltaxel, or a combination thereof.
